(19) United States
(12) Patent Application Publication
(10) Pub. No.: US 2019/0380740 A1
(43) Pub. Date: Dec. 19, 2019

(54) Guided Needle Actuation

(71) Applicant: Spiration, Inc. d/b/a Olympus Respiratory America, Redmond, WA (US)

(72) Inventors: Madeline C. Graham, Redmond, WA (US); Michael S. Smith, Sammamish, WA (US)

(21) Appl. No.: 16/007,208

(22) Filed: Jun. 13, 2018

(51) Int. Cl. A61B 17/34; A61B 10/02

(52) U.S. Cl. CPC A61B 17/3403; A61B 2017/00477; A61B 10/0233

(57) ABSTRACT

A medical device for controlling advancement of a needle or other active medical component. An exemplary device includes a needle, a sheath, and a handle. The handle includes an actuator component coupled to a proximal end of the needle and a guide component coupled to a proximal end of the sheath. The guide component slidably receives a portion of the actuator and the guide component allows the actuator to move distally via a plurality of groups of different positions, wherein each of the groups has a different radial value relative to the guide component. The guide component includes a track having a zig-zag pattern.

### BACKGROUND

Some of the current methods used for controlling needle/device advancement include using an O-ring mechanism that catches in grooves in the handle for slowing progress of the sliding component. This also provides tactile feedback to the user. Another mechanism uses a collar that can lock and unlock at desired locations along the handle shaft so that the sliding component cannot move past that lock.

### SUMMARY

The present invention includes an apparatus for controlling advancement of a needle or other medical device relative to a sheath or a catheter.

In one aspect of the invention, an exemplary apparatus includes a needle having a distal end and a proximal end, a sheath having a distal end and a proximal end and a handle. The handle includes an actuator component that is in mechanical communication with the needle and a guide component that is coupled to the proximal end of the sheath. The guide component slidably receives at least a portion of the actuator. Also, the guide component allows the actuator to move distally via a plurality of groups of different positions, wherein each of the groups has a different radial value relative to the guide component.

In another aspect of the invention, the actuator includes a handle portion and a shaft portion coupled to a distal end of the handle portion. The guide component slidably receives the shaft portion. A radial protrusion of the shaft portion extends beyond an outer diameter of the shaft portion. The guide component includes a first guide that allows motion of the radial protrusion between a first longitudinal position to a second longitudinal position, a second guide that allows motion of the radial protrusion between a third longitudinal position to a fourth longitudinal position, and a third guide that allows motion of the radial protrusion between a fifth longitudinal position to a sixth longitudinal position. Also, the guide component includes a seventh longitudinal position that is adjacent to the sixth longitudinal position, which restricts longitudinal motion of the actuator.

In still another aspect of the invention, the second longitudinal position and the third longitudinal position are equivalent in the longitudinal direction, the fourth longitudinal position and the fifth longitudinal position are equivalent in the longitudinal direction, and the sixth and seventh longitudinal positions are equivalent in the longitudinal direction.

In yet another aspect of the invention, the guide component includes a housing having a track formed in the housing. The track includes the first, second, and third guides. The first, second and third guides of the track are located at different radial positions on the housing. The track further includes a first cross-guide section that connects the first guide to the second guide, a second cross-guide section that connects the second guide to the third guide, and a third cross-guide section that connects the sixth longitudinal position to the seventh longitudinal position. The track may have a zig-zag pattern or curved or slanted paths.

In a further aspect of the invention, the longitudinal distance between the first longitudinal position and the second longitudinal position is equivalent to the longitudinal distance between the third longitudinal position and the fourth longitudinal position and the longitudinal distance between the fifth longitudinal position and the sixth longitudinal position.

In yet further aspects of the invention, the apparatus further includes a coupling device that couples the actuator to the needle. The coupling device decouples a rotational force applied to the actuator from translating to the needle.

Further features, advantages, and areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way. The components in the figures are not necessarily to scale, emphasis instead being placed upon illustrating the principles of the invention. In the drawings:

FIG. 1 shows a perspective view of a medical device formed in accordance with an embodiment of the present invention;

FIG. 2 shows a perspective view of a portion of the medical device of FIG. 1;

FIG. 3 shows another perspective view of the portion of the medical device of FIG. 2;

FIG. 4-1 shows proximal and distal features of the medical device of FIG. 1 at a first rotational section;

FIG. 4-2 shows proximal and distal features of the medical device of FIG. 1 at a second position;

FIG. 4-3 shows proximal and distal features of the medical device of FIG. 1 at a third rotational section;

FIG. 4-4 shows proximal and distal features of the medical device of FIG. 1 at a fourth rotational section; and

FIG. 5 shows a cross-section view of a handle portion and a side view of a proximal needle section of a medical device formed in accordance with an embodiment of the present invention.

### DETAILED DESCRIPTION

The following description is merely illustrative in nature and is not intended to limit the present disclosure, application, or uses. The following description explains, by way of illustration only and not of limitation, various embodiments of devices for controlling advancement of a needle or medical device into tissue relative to a sheath or a catheter, as well as systems including such devices and methods of using the same. It will be appreciated that various embodiments described herein may help to simplify the process of tissue aspiration.

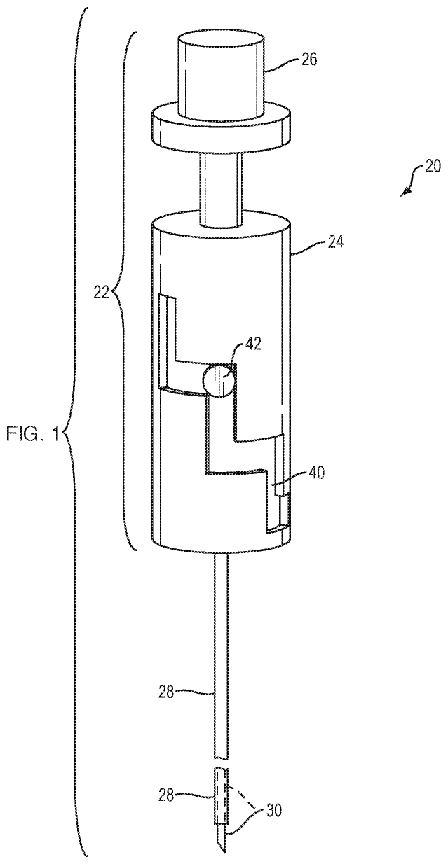

Referring to FIG. 1, a medical device 20 includes a proximal end having a handle 22 connected to a needle 30 (or other active medical device/tool) and a sheath 28. The needle 30 is slidably received within the sheath 28. The handle 22 includes a base section 24 and an actuator section 26. A distal end of the base section 24, is attached to a proximal end of the sheath 28. A distal end of the actuator section 26 receives and/or connects to a proximal end of the needle 30 within the base section 24. The base section 24 includes a guide or a track 40 that guides longitudinal and rotational movement of the actuator section 26 due to a protrusion device 42 that extends radially from a portion of the actuator section 26 that is located within the base section 24.

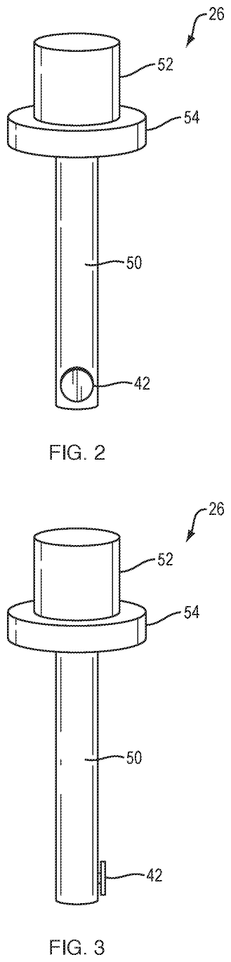

As shown in FIGS. 2 and 3, the actuator section 26 includes a shaft portion 50, a handle portion 52, and a handle stop portion 54. The handle stop portion 54 is located between a proximal end of the shaft portion 50 and a distal end of the handle portion 52. The protrusion device 42 is either monolithically part of the shaft portion 50 or is attached to the shaft portion 50. In one embodiment, the protrusion device 42 is a set pin or screw that attaches to or screws into a threaded lumen at the side of a distal end of the shaft portion 50. When the protrusion device 42 is properly mounted to the shaft portion 50, the protrusion device 42 extends away from a longitudinal axis of the actuator section 26 to at least beyond an inner diameter of the base section 24, such that motion of the actuator section 26 is controlled by the protrusion device 42 as it travels within the track 40.

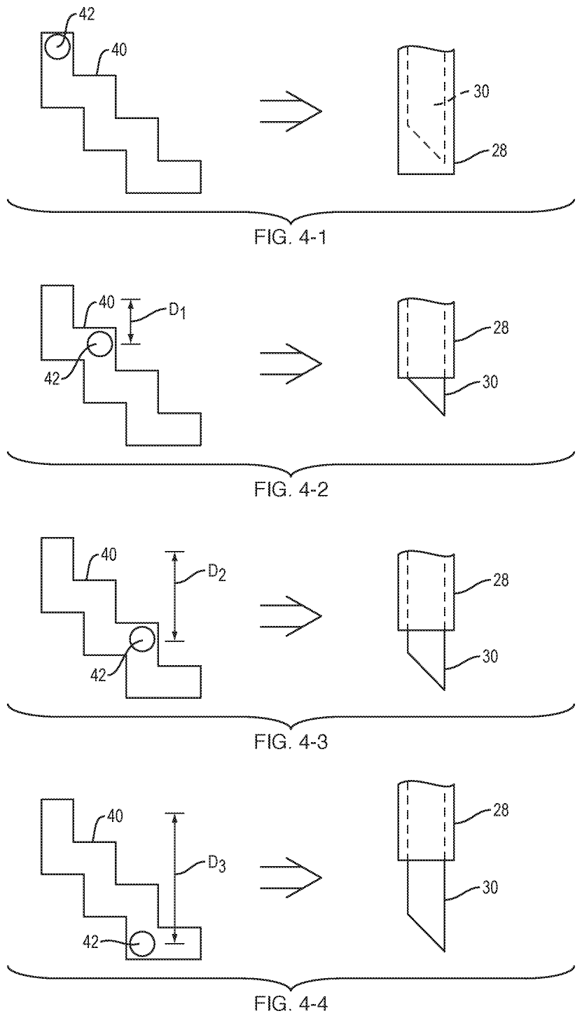

FIGS. 4-1 through 4-4 illustrate the locations of the protrusion device 42 within the track 40 and a corresponding position of the distal end of the needle 30 relative to the distal end of the sheath 28. The track 40 is a passageway that is cut through the surface of the base section 24. The track 40 may be created by any number of different methods, e.g., machining, laser etching, etc. The track 40 is a zigzag pattern that includes longitudinal sections that are parallel to a longitudinal axis of the handle 22 and horizontal or circumferential sections having tangential vectors that are perpendicular to the longitudinal axis. As shown in FIG. 4-1, the protrusion device 42 is shown in a most proximal position within the track 40. As such, the distal end of the needle 30 is fully retracted within the sheath 28. As shown in FIG. 4-2, the protrusion device 42 has been advanced to a first extended position that is a first longitudinal distance Di from the most proximal position shown in FIG. 4-1 and rotated to a rotational position that is a first rotational distance from the position shown in FIG. 4-1. In the first extended position the distal end of the needle 30 is slightly protruding from the distal end of the sheath 28. As shown in FIG. 4-3, the protrusion device 42 has been advanced to a second extended position that is a second longitudinal distance D2 from the most proximal position shown in FIG. 4-1 and rotated to a rotational position that is a second rotational distance from the position shown in FIG. 4-1. In the second extended position the distal end of the needle 30 is fully protruding from the distal end of the sheath 28. As shown in FIG. 4-4, the protrusion device 42 has been advanced to a third extended position that is a third longitudinal distance D3 from the most proximal position shown in FIG. 4-1. The protrusion device 42 may be rotated to a rotational position that is a third rotational distance from the position shown in FIG. 4-1. In the third extended position the distal end of the needle 30 is extended well beyond the distal end of the sheath 28. When the protrusion device 42 is rotated to the third rotational distance from the position shown in FIG. 4-1, the needle 30 will be held in place relative to the guide portion 24 when a force in the proximal direction is applied to the distal end of the needle 30. Guides with other shapes may be used for directing needle or other medical device deployment.

In one embodiment, each of the distances of the positions shown in FIGS. 4-2 through 4-4 are predefined to have equivalent longitudinal steps. For example, D₁ is equal to distance X, D₂ is equal to distance 2X and D₃ is equal to distance 3X. The rotation of the actuator section 26 would cause the needle 30 to rotate due to a direct connection between the needle 30 and the shaft portion 50. The twisting of the needle 30 into the tissue may improve sampling if used as a biopsy device, or it may just help improve penetration into dense tissue.

In one embodiment, detents or protrusions (not shown) are located at various points along the edge of the track 40. The detents or protrusions are sized and positioned to keep the protrusion device 42 in its current location within the track 40, unless the user applies a force greater than a predefined threshold in order to move the actuator section 26 relative to the track 40 and the base section 24.

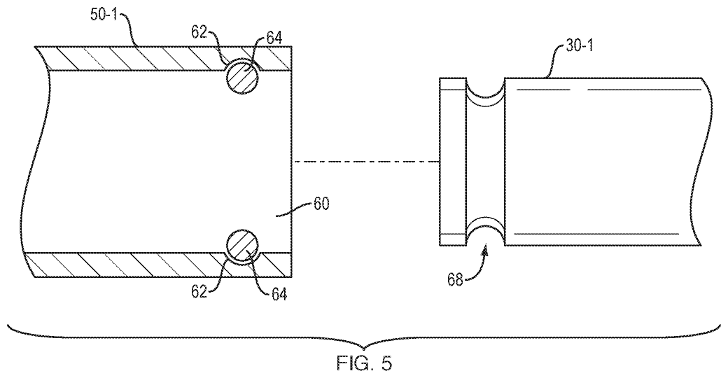

FIG. 5 illustrates a cross-sectional view of a distal end of a shaft portion 50-1 of an actuator and a side view of the proximal end of a needle 30-1. In one embodiment, an annular groove 68 is machined or formed on the outer surface of the needle 30-1 near the proximal end. The shaft portion 50-1 includes an annular groove 62 on the inner wall near the distal end. An O-ring 64 is at least partially received within the annular groove 62. When the proximal end of the needle 30-1 is received within the shaft portion 50-1, the O-ring 64 is also at least partially received within the annular groove 68 of the needle 30-1. The outer diameter of the needle 30-1 between the proximal end and the groove 68 may be smaller than the rest of the needle 30-1. This would make it easier to seat the needle 30-1 within the shaft portion 50-1.

In one embodiment, the annular groove 68 only extends around the needle 30-1 far enough to enable the set pin/screw 42 to move within the track 40 without causing rotational motion of the needle 30-1. In this embodiment, the O-ring 64 would be replaced by some other device (e.g., one or more ball bearing) that would allow for rotation of the shaft portion 50-1 relative to the needle 30-1.

In one embodiment, an annular slot is formed at least partially around the shaft portion. The annular slot receives a protrusion from the needle. The needle protrusion may be similar to the protrusion device 42 of the actuator section 26.

### Embodiments

A. A medical device comprising: a needle comprising a distal end and a proximal end; a sheath comprising a distal end and a proximal end; and a handle comprising: an actuator component being in mechanical communication with the needle; and a guide component being coupled to the proximal end of the sheath, wherein the guide component is configured to slidably receive at least a portion of the actuator, wherein the guide component is configured to allow the actuator to move distally via a plurality of groups of different positions, wherein each of the groups has a different radial value relative to the guide component.

B. The medical device of A, wherein the actuator comprises: a handle portion; a shaft portion coupled to a distal end of the handle portion, the shaft portion being slidably received within the guide component; and a radial protrusion that extends beyond an outer diameter of the shaft portion.

C. The medical device of B, wherein the guide component comprises: a first longitudinal guide configured to allow motion of the radial protrusion between a first longitudinal position to a second longitudinal position; a second longitudinal guide configured to allow motion of the radial protrusion between a third longitudinal position to a fourth longitudinal position; and a third longitudinal guide configured to allow motion of the radial protrusion between a fifth longitudinal position to a sixth longitudinal position.

D. The medical device of C, wherein the second longitudinal position and the third longitudinal position are equivalent in the longitudinal direction, wherein the fourth longitudinal position and the fifth longitudinal position are equivalent in the longitudinal direction.

E. The medical device of any of C or D, wherein the guide component comprises a housing having a track formed in the housing, wherein the track includes the first, second, and third guides.

F. The medical device of E, wherein the first, second, and third guides of the track are located at different radial positions on the housing.

G. The medical device of any of E or F, wherein the track further comprises: a first cross-guide section that connects the first guide to the second guide; a second cross-guide section that connects the second guide to the third guide; and a third cross-guide section that connects the sixth longitudinal position and a seventh longitudinal position.

H. The medical device of any of E-G, wherein the track has a zig-zag pattern.

I. The medical device of any of C-H, wherein the longitudinal distance between the first longitudinal position and the second longitudinal position is equivalent to the longitudinal distance between the third longitudinal position and the fourth longitudinal position.

J. The medical device of any of C-I, wherein the longitudinal distance between the first longitudinal position and the second longitudinal position is equivalent to the longitudinal distance between the fifth longitudinal position and the sixth longitudinal position.

K. The medical device of any of A-J, further comprising a coupling device configured to couple the actuator to the needle.

L. The medical device of K, wherein the coupling device decouples a rotational force applied to the actuator from translating to the needle.

M. The medical device of any of B-L, wherein the actuator further comprises a stop located between the handle portion and the shaft portion.

N. The medical device of any of A-M, wherein the sheath is configured to slidably receive the needle.

O. A medical device comprising: a needle comprising a distal end and a proximal end; a sheath comprising a distal end and a proximal end; and a handle comprising: an actuator component being in mechanical communication with the needle, the actuator comprising: a handle portion; a stop attached to the handle portion; a shaft attached to the stop and the proximal end of the needle; and a protrusion device removably attached to the shaft; and a housing comprising a zig-zag pattern slot exposing an inner lumen to an exterior environment, the zig-zag pattern slot comprises a plurality of longitudinal slots and a plurality of circumferential slots connected between longitudinal slots, wherein the zig-zag pattern slot is configured to guide motion of the protrusion device and thus the actuator.

P. The medical device of 0, further comprising a coupling device configured to couple the actuator to the needle.

Q. The medical device of P, wherein the coupling device decouples a rotational force applied to the actuator from translating to the needle.

R. The medical device of any of O-Q, wherein the sheath is configured to slidably receive the needle.

The description of the invention is merely exemplary in nature and variations that do not depart from the gist of the invention are intended to be within the scope of the invention. Such variations are not to be regarded as a departure from the spirit and scope of the invention.

Although the preferable embodiments of the present invention have been described hitherto, the present invention is not limited to these embodiments. Additions, omissions, substitutions, and other modifications can be made without departing from the spirit or scope of the present invention.

In addition, the invention is not limited by the above description and is limited by only the scope of appended claims.

What is claimed is:

1. A medical device comprising:
a medical tool comprising a distal end and a proximal end;
a sheath comprising a distal end and a proximal end; and
a handle comprising:
an actuator component being in mechanical communication with the medical tool; and
a guide component being coupled to the proximal end of the sheath, wherein the guide component is configured to slidably receive at least a portion of the actuator, wherein the guide component is configured to allow the actuator to move distally via a plurality of groups of different positions, wherein each of the groups has a different radial value relative to the guide component.

2. The medical device of claim 1, wherein the actuator comprises:
a handle portion;
a shaft portion coupled to a distal end of the handle portion, the shaft portion being slidably received within the guide component; and
a radial protrusion that extends beyond an outer diameter of the shaft portion.

3. The medical device of claim 2, wherein the guide component comprises:
a first longitudinal guide configured to allow motion of the radial protrusion between a first longitudinal position to a second longitudinal position;
a second longitudinal guide configured to allow motion of the radial protrusion between a third longitudinal position to a fourth longitudinal position; and
a third longitudinal guide configured to allow motion of the radial protrusion between a fifth longitudinal position to a sixth longitudinal position.

4. The medical device of claim 3, wherein the second longitudinal position and the third longitudinal position are equivalent in the longitudinal direction, wherein the fourth longitudinal position and the fifth longitudinal position are equivalent in the longitudinal direction.

5. The medical device of claim 3, wherein the guide component comprises a housing having a track formed in the housing, wherein the track includes the first, second, and third guides.

6. The medical device of claim 5, wherein the first, second, and third guides of the track are located at different radial positions on the housing.

7. The medical device of claim 6, wherein the track further comprises:
a first cross-guide section that connects the first guide to the second guide;
a second cross-guide section that connects the second guide to the third guide; and
a third cross-guide section that connects the sixth longitudinal position and a seventh longitudinal position.

8. The medical device of claim 7, wherein the track has a zig-zag pattern.

9. The medical device of claim 8, wherein the longitudinal distance between the first longitudinal position and the second longitudinal position is equivalent to the longitudinal distance between the third longitudinal position and the fourth longitudinal position.

10. The medical device of claim 9, wherein the longitudinal distance between the first longitudinal position and the second longitudinal position is equivalent to the longitudinal distance between the fifth longitudinal position and the sixth longitudinal position.

11. The medical device of claim 1, further comprising a coupling device configured to couple the actuator to the medical tool.

12. The medical device of claim 11, wherein the coupling device decouples a rotational force applied to the actuator from translating to the medical tool.

13. The medical device of claim 2, wherein the actuator further comprises a stop located between the handle portion and the shaft portion.

14. The medical device of claim 1, wherein the sheath is configured to slidably receive the medical tool.

15. The medical device of claim 1, wherein the medical tool comprises a needle.

16. A medical device handle comprising:
an actuator component being mechanically connectable with a medical tool, the actuator comprising:
a handle portion;
a stop attached to the handle portion;
a shaft attached to the stop and attachable to a proximal end of the medical tool; and
a protrusion device removably attached to the shaft; and
a housing comprising a zig-zag pattern slot exposing an inner lumen to an exterior environment, the zig-zag pattern slot comprises a plurality of longitudinal slots and a plurality of circumferential slots connected between longitudinal slots,
wherein the zig-zag pattern slot is configured to guide motion of the protrusion device and thus the actuator.

17. A handle for a medical device comprising:
an actuator component being mechanically connectable a medical tool; and
a guide component being connectable to a proximal end of a sheath, wherein the guide component is configured to slidably receive at least a portion of the actuator, wherein the guide component is configured to allow the actuator to move distally via a plurality of groups of different positions, wherein each of the groups has a different radial value relative to the guide component.

18. The handle of claim 17, wherein the actuator comprises:
a handle portion;
a shaft portion coupled to a distal end of the handle portion, the shaft portion being slidably received within the guide component; and
a radial protrusion that extends beyond an outer diameter of the shaft portion.

19. The handle of claim 18, wherein the guide component comprises:
a first longitudinal guide configured to allow motion of the radial protrusion between a first longitudinal position to a second longitudinal position;
a second longitudinal guide configured to allow motion of the radial protrusion between a third longitudinal position to a fourth longitudinal position; and
a third longitudinal guide configured to allow motion of the radial protrusion between a fifth longitudinal position to a sixth longitudinal position.

20. The handle of claim 19, wherein the second longitudinal position and the third longitudinal position are equivalent in the longitudinal direction, wherein the fourth longitudinal position and the fifth longitudinal position are equivalent in the longitudinal direction,
wherein the guide component comprises a housing having a track formed in the housing,
wherein the track includes the first, second, and third guides,
wherein the first, second, and third guides of the track are located at different radial positions on the housing,
wherein the track further comprises:
a first cross-guide section that connects the first guide to the second guide;
a second cross-guide section that connects the second guide to the third guide; and
a third cross-guide section that connects the sixth longitudinal position and a seventh longitudinal position.
